(19) United States
(12) Patent Application Publication
(10) Pub. No.: US 2019/0175484 A1
(43) Pub. Date: Jun. 13, 2019

(54) RHEOLOGY MODIFICATION OF PERSONAL CARE COMPOSITIONS

(71) Applicant: Dow Global Technologies LLC, Midland, MI (US)

(72) Inventors: Muriel Cuilleret, Grasse (FR); Frederic De La Torre, Antibes (FR)

(21) Appl. No.: 16/099,730

(22) PCT Filed: Jun. 16, 2017
(86) PCT No.: PCT/US2017/037836

§ 371 Date: Nov. 8, 2018

(30) Foreign Application Priority Data
Jun. 23, 2016 (EP) 16290114.4
Oct. 24, 2016 (EP) 16290206.8

(51) Int. Cl. A61K 8/81; A61K 8/19; A61K 8/41; A61Q 5/00; A61Q 19/00

(52) U.S. Cl. CPC A61K 8/8152; A61K 8/19; A61Q 19/00; A61Q 5/00; A61K 8/41

(57) ABSTRACT

Provided are personal care compositions comprising personal care composition comprising (a) carbon dioxide, (b) an ionic acrylic based rheology modifier comprising at least one of an alkali swellable emulsion polymer and a hydrophobically-modified alkali swellable emulsion polymer, and (c) a neutralizer. Also provided are methods for altering over time the viscosity of a personal care composition comprising a rheology modifier and a neutralizer after release from a container in which the composition is stored, comprising (a) dissolving carbon dioxide in the composition in an enclosed container in an amount of from 0.5 to 10 weight %, based on the total weight of the composition, and (b) topically administering an effective amount of the personal care composition from the enclosed container onto the skin or hair, wherein the rheology modifier comprises at least one of an alkali swellable emulsion polymer and a hydrophobically modified alkali swellable emulsion polymer, whereby after release from the container the viscosity of the composition changes as a function of a change in pH as dissolved carbon dioxide escapes.

### FIELD OF THE INVENTION

This invention relates generally to personal care formulations containing rheology modifiers and carbon dioxide. The rheology modifiers are ionic and demonstrate changes in viscosity as a function of pH.

### BACKGROUND

Personal care compositions contain a variety of additives that provide a wide array of benefits to the composition. One class of additives are thickeners that provide viscosity enhancements and impart good aesthetics, such as good sensory feel. One type of thickening agent known in the art are ionic in nature. These thickeners, however, do not have the ability on their own to change viscosity after application to the skin or hair except under shear.

The prior art has utilized the inclusion of gases in self-foaming cosmetic or dermatological preparations. For example, US 2004/0241105 A1 such compositions comprising an emulsifier system, a lipid phase, an inorganic thickener, a hydrocolloid, a particulate hydrophobic, hydrophobicized or oil-absorbing solid-body substance, and at least one gas selected from the group consisting of air, oxygen, nitrogen, helium, argon, nitrous oxide, and carbon dioxide. The prior art does not, however, disclose a personal care composition according to the present invention which allows for a change in sensorial feel from a low to high viscosity formulation after release from packaging for application to the skin or hair.

Accordingly, there is a need to develop compositions containing thickeners that demonstrate the ability to change from a liquid-like low viscosity sensorial feel to a gel-like high viscosity sensorial feel after release from packaging for application to the skin or hair.

### STATEMENT OF INVENTION

One aspect of the invention provides a personal care composition comprising (a) carbon dioxide, (b) an ionic acrylic based rheology modifier comprising at least one of an alkali swellable emulsion polymer and a hydrophobically-modified alkali swellable emulsion polymer, and (c) a neutralizer. In certain embodiments, the neutralizer comprises at least one of sodium hydroxide, potassium hydroxide, sodium carbonate, potassium carbonate, monoethanolamine, triethanolamine, aminomethylpropanol, aminomethylpropanediol, and tromethamine. In certain embodiments, the rheology modifier comprises a compound of Formula (I):

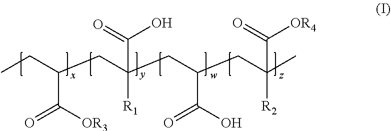

Wherein R₁ and R₂ are each independently H or a C₁-C₆ alkyl group, and R₃ and R₄ are each independently H or a C₁-C₂₄ alkyl or alkoxylated alkyl group.

Another aspect of the invention provides a method for altering over time the viscosity of a personal care composition comprising a rheology modifier and a neutralizer after release from a container in which the composition is stored, comprising (a) dissolving carbon dioxide in the composition in an enclosed container in an amount of from 0.5 to 10 weight %, based on the total weight of the composition, and (b) topically administering an effective amount of the personal care composition from the enclosed container onto the skin or hair, wherein the rheology modifier comprises at least one of an alkali swellable emulsion polymer and a hydrophobically modified alkali swellable emulsion polymer, whereby after release from the container the viscosity of the composition changes as a function of a change in pH as dissolved carbon dioxide escapes.

### DETAILED DESCRIPTION

The inventors have now surprisingly found that personal care compositions comprising carbon dioxide and an ionic acrylic based rheology modifier provide the ability to alter the viscosity of the composition after deposition onto the skin or hair. While not wishing to be bound by theory, it is believed that the introduction of carbon dioxide in formulations including acrylic based ionic rheology modifiers creates a modification of the acidity/pH that impacts the rheological behavior of the formulation. For example, a gel-like formulation can change to a liquid-like formulation upon lowering of the pH by introduction of the carbon dioxide in an enclosed container. Once the composition is removed from the enclosed container for administration onto the skin or hair, the dissolved carbon dioxide will be released from the formulation, thereby raising the pH of the composition and changing the formulation from a liquid-like low viscosity formulation back to a gel-like high viscosity formulation.

In the present invention, “personal care” is intended to refer to cosmetic and skin care compositions for application to the skin, including, for example, body washes and cleansers, as well as leave on application to the skin, such as lotions, creams, gels, gel creams, serums, toners, wipes, liquid foundations, make-ups, tinted moisturizer, oils, face/body sprays, and topical medicines. In the present invention, “personal care” is also intended to refer to hair care compositions including, for example, shampoos, leave-on conditioners, rinse-off conditioners, styling gels, pomades, hair coloring products (e.g., two-part hair dyes), hairsprays, and mousses. Preferably, the personal care composition is cosmetically acceptable. “Cosmetically acceptable” refers to ingredients typically used in personal care compositions, and is intended to underscore that materials that are toxic when present in the amounts typically found in personal care compositions are not contemplated as part of the present disclosure. The compositions of the invention may be manufactured by processes well known in the art, for example, by means of conventional mixing, dissolving, granulating, emulsifying, encapsulating, entrapping or lyophilizing processes.

As used herein, the term “polymer” refers to a polymeric compound prepared by polymerizing monomers, whether of the same or a different type. The generic term “polymer” includes the terms “homopolymer,” “copolymer,” and “terpolymer.” As used herein, the term “polymerized units derived from” refers to polymer molecules that are synthesized according to polymerization techniques wherein a product polymer contains “polymerized units derived from” the constituent monomers which are the starting materials for the polymerization reactions. As used herein, the term “(meth)acrylate” refers to either acrylate or methacrylate, and the term “(meth)acrylic” refers to either acrylic or methacrylic. As used herein, the term “substituted” refers to having at least one attached chemical group, for example, alkyl group, alkenyl group, vinyl group, hydroxyl group, carboxylic acid group, other functional groups, and combinations thereof.

The inventive personal care compositions include dissolved carbon dioxide. As noted above, while not wishing to be bound by theory, it is believed that the introduction of carbon dioxide in formulations including acrylic based ionic rheology modifiers creates a modification of the acidity/pH that impacts the rheological behavior of the formulation. In certain embodiments, the carbon dioxide is introduced into the composition in an amount sufficient to reduce the pH of a composition by several pH units. In certain embodiments, the carbon dioxide is present in an amount of from 0.5 to 10 weight %, preferably of from 1.0 to 5.0 weight %, and more preferably of from 1.5 to 3.0 weight %, based on the total weight of the composition.

The inventive personal care compositions also include an ionic rheology modifier. Suitable ionic rheology modifiers include, for example, synthetic polymers with amine groups, acid groups, or both. Such synthetic polymers include, for example, polymers made from monomer mixtures that include one or more amine-containing (meth)acrylate monomers (such as, for example, (meth)acrylamide) and, optionally other monomers, including, for example, one or more non-amine-containing (meth)acrylate monomers. Synthetic polymers suitable as ionic rheology modifiers also include, for example, polymers made from monomer mixtures that include one or more acid-containing (meth)acrylate monomers (such as, for example, (meth)acrylic acid) and, optionally other monomers, including, for example, one or more non-acid-containing (meth)acrylate monomers (which may be monoethylenically unsaturated or multiethylenically unsaturated or a mixture thereof).

Other suitable ionic rheology modifiers include, for example, hydrophobically-modified synthetic polymers made from monomer mixtures that include at least one amine-containing monomer or at least one acid-containing monomer. The hydrophobic group may be attached, for example, by including, in the monomer mixture from which the hydrophobically modified synthetic polymer is made, one or more monomers with hydrophobic groups (i.e., a hydrocarbons with 6 or more carbon atoms, fluoro-substituted hydrocarbons with 3 or more carbon atoms and at least one fluorine atom, organosiloxane-containing organic radicals, or combinations thereof); such polymers include, for example, copolymers made from monomer mixtures that include (meth)acrylic acid, C₁₀-C₂₀ alkyl (meth)acrylates, optionally multiethylenically unsaturated (meth)acrylates, and other (meth)acrylates.

In certain embodiments, the ionic rheology modifier comprises at least one of an alkali swellable emulsion (“ASE”) polymer and a hydrophobically-modified alkali swellable emulsion (“HASE”) polymer. ASE and HASE polymers are typically synthesized by free-radical emulsion polymerization of varying mixtures of hydrophilic monomers such as acrylic acid, methacrylic acid, or maleic anhydride, lipophilic monomers such as ethyl acrylate, butyl acrylate, or methyl methacrylate, and associative monomers such as long chain (C₈-C₂₂) acrylates or styrenic derivatives. Preferred ASE and HASE polymers have the structure of Formula (I):

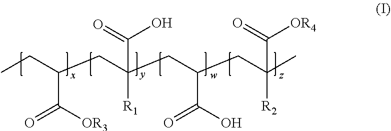

wherein: R₁ and R₂ are each independently H or a C₁-C₆ alkyl group; R₃ and R₄ are each independently H or a C₁-C₂₄ alkyl or alkoxylated alkyl group; and x, y, w, and z represent the percentage by weight that each repeating unit or derived monomer is contained within the polymer and add up to a total of 100 weight % of the total weight of the polymer, and x is from about 30 to 60 weight %, y is from 10 to 50 weight %, w is from 0 to 25 weight %, and z is from 0 to 25 weight %.

In certain embodiments, each of R₁, R₂, R₃, and R₄ are H.

In certain embodiments, R₁ is an alkyl group, preferably a C₁-C₆ alkyl group, more preferably a C₁ or C₂ alkyl group, and even more preferably a C₁ alkyl group.

In certain embodiments, R₂ is an alkyl group, preferably a C₁-C₆ alkyl group, more preferably a C₁ or C₂ alkyl group, and even more preferably a C₁ alkyl group.

In certain embodiments, R₃ is any suitable hydrophobic group, such as a C₁-C₂₄ alkyl group, and more preferably a C₈-C₂₀ alkyl group. In certain embodiments, R₃ is C₁-C₂₄ alkoxylated alkyl group, preferably ethoxylated or propoxylated. When alkoxylated, R₃ can be alkoxylated to a degree of from 1 to 60, preferably of from 10 to 50.

In certain embodiments, R₄ is any suitable hydrophobic group, such as a C₁-C₂₄ alkyl group, and more preferably a C₈-C₂₀ alkyl group. In certain embodiments, R₄ is C₁-C₂₄ alkoxylated alkyl group, preferably ethoxylated or propoxylated. When alkoxylated, R₄ can be alkoxylated to a degree of from 1 to 60, preferably of from 10 to 50.

In certain embodiments, R₁ and R₂ are each independently a C₁-C₆ alkyl group, and R₃ and R₄ are each independently a C₁-C₂₄ alkyl or alkoxylated alkyl group.

The ratio of x:y can be from 1:20 to 20:1, preferably from 1:10 to 10:1, and more preferably from 1:5 to 5:1. The ratio of x:w can be from 1:20 to 20:1, preferably from 1:10 to 10:1, and more preferably from 1:5 to 5:1. The ratio of x:z can be from 1:1 to 500:1, preferably from 2:1 to 250:1, and more preferably from 25:1 to 75:1.

In certain embodiments, the polymer of the present invention is crosslinked, that is, at least one crosslinker, such as a monomer having two or more ethylenic unsaturated groups, is included with the copolymer components of Formula (I) during polymerization. Suitable crosslinkers include, for example, divinylbenzene, trimethylolpropane diallyl ether, tetraallyl pentaerythritol, triallyl pentaerythritol, diallyl pentaerythritol, diallyl phthalate, triallyl cyanurate, Bisphenol A diallyl ether, methylene bisacrylamide and allyl sucroses. In certain embodiments, the crosslinker does not have ester or amide functionality. In certain preferred embodiments, the crosslinker comprises one or more of divinylbenzene, trimethylolpropane diallyl ether (TMPDE) and tetraallyl pentaerythritol. The amount of crosslinker residue in the polymer is from 0.01% to 3%, based on the total weight of the monomers. Preferably, the amount of crosslinker residue in the polymer is no more than 1%, more preferably no more than 0.5%, and even more preferably no more than 0.2%. Preferably, the amount of crosslinker residue in the polymer is at least 0.05%, more preferably at least 0.1%.

Polymer molecular weights can be measured by standard methods such as, for example, size exclusion chromatography or intrinsic viscosity. In certain embodiments, the ASE and HASE polymers of the present invention have a weight average molecular weight (M_{w}) of from 50,000 to 1,000,000 g/mol, preferably of from 80,000 to 500,000 g/mol, and more preferably of from 100,000 to 300,000 g/mol, as measured by gel permeation chromatography.

Suitable ASE polymers include, for example, ACULYN 33 (available from The Dow Chemical Company) and ACULYN 38 (available from The Dow Chemical Company). Suitable HASE polymers include, for example, ACULYN 22 (available from The Dow Chemical Company), ACULYN 28 (available from The Dow Chemical Company), ACULYN 88 (available from The Dow Chemical Company), and ACULYN EXCEL (available from The Dow Chemical Company).

Suitable polymerization techniques for preparing the particles contained in the inventive skin care compositions include, for example, miniemulsion polymerization, as described, e.g., in G. H. Al-Ghamdi et al., J. Appl. Poly. Sci., vol. 101, pp. 3479-3486 (2006), or Landfester, K., Macromol. Rapid. Commun., vol. 22, pp. 896-936 (2001), and references cited therein, using an ultrasonic or high-pressure homogenizer, preferably with sufficient power to create 200 nm droplets. Aqueous emulsion polymerization processes typically are conducted in an aqueous reaction mixture, which contains at least one monomer and various synthesis adjuvants, such as the free radical sources, buffers, and reductants in an aqueous reaction medium. In certain embodiments, a chain transfer agent may be used to limit molecular weight. The aqueous reaction medium is the continuous fluid phase of the aqueous reaction mixture. Surfactants suitable for conventional emulsion polymerization are also suitable for miniemulsion polymerization. In certain embodiments, the polymerization is performed at temperatures in the range of from 20 to 100° C. In certain embodiments, the solids content of the polymerization mixture is in a range of from 20 to 60 weight %, preferably from 20 to 60 weight %, more preferably from 25 to 55 weight %, and even more preferably from 30 to 50 weight %. In certain embodiments, the aqueous reaction medium has a pH of less than or equal to 5, and preferably having a pH of less than or equal to 5. Methods of making such ASE and HASE polymers are described in U.S. Pat. Nos. 4,514,552, 5,192,592, British Patent No. 870,994, and U.S. Pat. No. 7,217,443.

In certain embodiments, the composition contains additional rheology modifiers including, for example, carbomer (e.g., CARBOPOL 980 available from Lubrizol), polyacrylate-1 crosspolymer (e.g., CARBOPOL Aqua CC available from Lubrizol), acrylates crosspolymer-4 (e.g., CARBOPOL SF-2 available from Lubrizol), ammonium acryloyldimethyltaurate/VP copolymer (e.g., ARISTOFLEX AVC available from Clariant), acrylates/C₁₀-C₃₀ alkyl acrylate crosspolymer (e.g., CARBOPOL Ultrez 21 available from Lubrizol), polyacrylamide and C₁₃-C₁₄ isoparaffin and laureth-7 (e.g., SEPPIGEL 305 available from Seppic), carboxylated or sulfated polysaccharides (e.g., carboxymethylcellulose), carboxylated or sulfated guar, carboxylated or sulfated carrageenan, carboxylated or sulfated xanthan, and alginates.

The rheology modifier is present in the personal care composition in an amount sufficient to impart the desired rheology profile to the desired composition, and is dependent upon the specific rheology modifier. By way of non-limiting example, the amount of rheology modifier (actives) in the composition of the invention may be in the range of from 0.1 to 10 weight %, preferably of from 0.2 to 5 weight %, and more preferably of from 0.3 to 3 weight %, based on the total weight of the composition.

The inventive personal care compositions also include a neutralizer which acts to neutralize the charge of the ionic acrylic rheology modifier. Suitable neutralizers include, for example, sodium hydroxide, potassium hydroxide, sodium carbonate, potassium carbonate, monoethanolamine, triethanolamine, aminomethylpropanol, aminomethylpropanediol, and tromethamine. In certain preferred embodiments, the neutralizer comprises at least one of sodium hydroxide, potassium hydroxide, monoethanolamine, triethanolamine, aminomethylpropanol, monoisopropanolamine, and tetrahydroxypropyl ethylenediamine. In certain embodiments, the neutralizer is present in an amount sufficient to completely or partially neutralize the rheology modifiers in the composition as measured in an environment in which carbon dioxide is substantially absent, e.g., prior to addition of carbon dioxide into the composition, and after carbon dioxide is released from the composition once the composition is removed from its container. By way of non-limiting example, the amount of neutralizer is present in the composition in an amount sufficient to impart a pH of from 2 to 13, preferably from 4 to 11, and more preferably from 5 to 9, as measured in an environment in which carbon dioxide is substantially absent from the composition.

The inventive personal care compositions also include a dermatologically acceptable carrier. Such material is typically characterized as a carrier or a diluent that does not cause significant irritation to the skin and does not negate the activity and properties of active agent(s) in the composition. Examples of dermatologically acceptable carriers that are useful in the invention include, without limitation, water, such as deionized or distilled water, emulsions, such as oil-in-water or water-in-oil emulsions, alcohols, such as ethanol, isopropanol or the like, glycols, such as propylene glycol, glycerin or the like, creams, aqueous solutions, oils, ointments, pastes, gels, lotions, milks, foams, suspensions, powders, or mixtures thereof. The aqueous solutions may contain cosolvents, e.g., water miscible cosolvents. Suitable water miscible cosolvents include, for example, ethanol, propanol, acetone, ethylene glycol ethyl ethers, propylene glycol propyl ethers, and diacetone alcohol. In some embodiments, the composition contains from about 99.99 to about 50 percent by weight of the dermatologically acceptable carrier, based on the total weight of the composition.

Other additives may be included in the compositions of the invention such as, but not limited to, surfactants, abrasives, absorbents, aesthetic components such as fragrances, pigments, colorings/colorants, essential oils, skin sensates, astringents (e.g., clove oil, menthol, camphor, eucalyptus oil, eugenol, menthyl lactate, witch hazel distillate), preservatives, anti-caking agents, a foam building agent, antifoaming agents, antimicrobial agents (e.g., iodopropyl butylcarbamate), antioxidants, binders, biological additives, buffering agents, bulking agents, chelating agents, chemical additives, cosmetic astringents, cosmetic biocides, denaturants, drug astringents, external analgesics, film formers or materials, e.g., polymers, for aiding the film-forming properties and substantivity of the composition (e.g., copolymer of eicosene and vinyl pyrrolidone), opacifying agents, pH adjusters, propellants, reducing agents, sequestrants, skin bleaching and lightening agents (e.g., hydroquinone, kojic acid, ascorbic acid, magnesium ascorbyl phosphate, ascorbyl glucosamine), skin-conditioning agents (e.g., humectants, including miscellaneous and occlusive), skin soothing and/or healing agents (e.g., panthenol and derivatives (e.g., ethyl panthenol), aloe vera, pantothenic acid and its derivatives, allantoin, bisabolol, and dipotassium glycyrrhizinate), skin treating agents, vitamins (e.g., Vitamin C) and derivatives thereof, silicones, and fatty alcohols. The amount of optional ingredients effective for achieving the desired property provided by such ingredients can be readily determined by one skilled in the art.

As noted above, personal care compositions of the present invention surprisingly allow for a changing rheology profile after release from the container in which it is packaged, for deposition onto the skin or hair. Thus, in one aspect the present invention provides a method for altering over time the viscosity of a personal care composition comprising a rheology modifier after release from the container in which it is packaged for application to the skin or hair comprising dissolving carbon dioxide in the composition in an enclosed container, and then topically administering an effective amount of the personal care composition from the enclosed container onto the skin or hair. In certain embodiments, the composition contains an ionic acrylic rheology modifier, preferably at least one of an alkali swellable emulsion polymer and a hydrophobically modified alkali swellable emulsion polymer. The carbon dioxide can be dissolved in the composition by any suitable technique known in the art; for example, carbon dioxide may be dissolved in the composition by use of a carbonation device that carbonates water by adding carbon dioxide from a pressurized cylinder, or by bubbling of carbon dioxide into pressurized mixing equipment. Suitable carbonation devices are available from SodaStream USA Inc. In practicing the methods of the invention, the personal care compositions are generally administered topically by applying, spreading, or spraying the compositions onto the skin or hair.

Some embodiments of the invention will now be described in detail in the following Examples.

### EXAMPLES

### Example 1

### Preparation of Exemplary Personal Care Formulations Prior to Carbonation

Exemplary personal care formulations according to the present invention contain the components recited in Table 1.

TABLE 1
Exemplary Personal Care Formulations Prior to Carbonation

| | | | | | | |
|---|---|---|---|---|---|---|
| | | E1 | E2 | E3 | E4 | E5 |
| Trade Name | INCI Name | (w/w %) | (w/w %) | (w/w %) | (w/w %) | (w/w %) |

Phase A

| | | | | | | |
|---|---|---|---|---|---|---|
| Water | — | 95.50 | 92.14 | 94.59 | 91.99 | 85.11 |
| ACULYN 28¹ | Acrylates/Beheneth- | 2.44 | 4.94 | 3.08 | — | — |
| (19.81% solids) | 25 Methacrylate | | | | | |
| rheology | Copolymer (HASE) | | | | | |
| modifier | | | | | | |
| ACULYN 33¹ | Acrylates | — | — | — | 4.90 | 9.66 |
| (28.30% solids) | Copolymer (ASE) | | | | | |
| rheology | | | | | | |
| modifier | | | | | | |

Phase B

| | | | | | | |
|---|---|---|---|---|---|---|
| NaOH (10%) | NaOH | 0.65 | 1.48 | 0.96 | 1.73 | 3.82 |
| Citric acid | Citric acid | 0.05 | — | — | — | — |

Phase C

| | | | | | | |
|---|---|---|---|---|---|---|
| Neolone PH100 | Phenoxyethanol | 0.83 | 0.63 | 0.59 | 0.79 | 0.79 |
| preservative¹ | | | | | | |
| Hydrolite 8 | Caprylyl Glycol | 0.53 | 0.81 | 0.78 | 0.59 | 0.63 |
| humectant | | | | | | |

| | | | | | |
|---|---|---|---|---|---|
| Total= | 100.00 | 100.00 | 100.00 | 100.00 | 100.00 |

¹Available from The Dow Chemical Company

Exemplary personal care formulations prior to carbonation E1 was prepared by mixing all of the Phase A components in a plastic beaker using a standard stirrer until homogenous. The Phase B components were then added to raise the pH to 7.85. The Phase C components were then added and the mixture was stirred until all components were homogenized and displayed a stable pH. The formulation was observed to be of a gel-like consistency.

Exemplary formulations E2-E5 were prepared substantially as described above, with the appropriate changes in amounts of each of the components as recited in Table 1.

### Example 2

### Carbonation of Exemplary Personal Care Formulations

Exemplary personal care formulations as prepared in Example 1 were carbonated with carbon dioxide according to the following procedures. Example personal care formulations E1-E5 were centrifuged in 4 bottles of 250 mL for 12 minutes at 2,000 rpm. Each formulation was then transferred to a 500 mL SodaStream container available from SodaStream USA Inc. Carbon dioxide was then introduced into each SodaStream container using a SodaStream carbonation device available from SodaStream USA Inc. until the automatic stop of the device was triggered. Prior to introduction of the carbon dioxide, the formulation displayed a high viscosity gel-like rheology profile. Upon introduction of the carbon dioxide into the top of the container, the formulation was observed to display a low viscosity liquid-like rheology profile that changed to a high viscosity gel-like rheology profile as a function of distance from the top of the container where the carbon dioxide was introduced. The contents of the container was manually shaken to allow the dissolved carbon dioxide to mix throughout the contents of the container. The above procedure was repeated twice after the initial introduction of carbon dioxide.

After the final loading of carbon dioxide, the contents of the container were allowed to “relax” for about 15 minutes to ensure that the gaseous carbon dioxide was fully dissolved in the liquid phase of the formulation, after which the formulation displayed a low viscosity liquid-like rheology profile throughout the contents of the container.

The formulation was thereafter transferred to a tightly sealed 200 mL pump spray. After spraying onto a surface the formulation was observed to quickly (in less than 10 seconds) thicken from a low viscosity liquid-like rheology profile to a high viscosity gel-like rheology profile similar to that displayed by the formulation prior to introduction of the carbon dioxide.

The quantity of gaseous carbon dioxide introduced into the formulations was be assessed by weighing the carbon dioxide gas cartridge utilized in the SodaStream carbonation device before and after carbon dioxide introduction into the formulations, as shown in Table 2.

TABLE 2
Amount of Dissolved Carbon Dioxide
in Formulation after Carbonation

| Sample | CO₂ in Formulation (w/w %) |
|---|---|
| E2 | 2.65 |
| E4 | 1.91 |
| E5 | 1.59 |

### Example 3

pH of Exemplary Personal Care Formulations E1-E3 Before and after Carbonation

The pH of each of Exemplary Formulations E1-E3 (containing HASE rheology modifiers) as prepared in Example 1 was measured before and after carbonation in Example 2. To visualize the change in pH, 10 droplets of Phenol Red (available from Sigma-Aldrich) were added to the formulation prior to carbonation, at which time the formulation displayed a pink color. After carbonation as described in Example 2, the formulation changed to yellow/clear color indicative of a drop in pH. The pH was measured using a SCHOTT CG837 pH meter. The pH and observable rheology profile of each of Example Formulations E1-E3 before and after carbonation is shown in Table 3.

TABLE 3
pH and Rheology Profile of Exemplary
Formulations Containing HASE Polymers

| | | | |
|---|---|---|---|
| SAMPLE | E1 | E2 | E3 |

Prior to Carbonation

| | | | |
|---|---|---|---|
| pH | 7.7 | 7.0 | 7.4 |
| Appearance | clear gel | clear thick gel | pink hazy |
| | | | thick gel |

After Carbonation

| | | | |
|---|---|---|---|
| pH | 5.8 | 5.2 | 5.5 |
| Appearance | clear liquid | clear low | Yellow clear |
| | | viscosity gel | thick liquid |

2 hrs After Carbonation (open bottle)

| | | | |
|---|---|---|---|
| pH | 6.3 | — | — |
| Appearance | clear gel | — | — |

24 hrs After Carbonation (open bottle)

| | | | |
|---|---|---|---|
| pH | — | — | 5.6 |
| Appearance | — | — | pink on top |
| | | | layer, yellow |
| | | | below surface |

24 hrs After Carbonation (closed bottle)

| | | | |
|---|---|---|---|
| pH | — | 5.8 | — |
| Appearance | — | — | — |

24 hrs After Carbonation (closed bottle) + 6 hrs (open bottle)

| | | | |
|---|---|---|---|
| pH | — | 6.0 | — |
| Appearance | — | — | — |

As demonstrated by the results in Table 3, the pH and rheology profile of the formulations changed from a high viscosity gel-like formulation to a low viscosity liquid-like formulation after introduction of carbon dioxide and reduction of pH, and then reversed back to a high viscosity gel-like formulation as the carbon dioxide was allowed to escape and the pH of the formulation returned to the pre-carbonation level.

### Example 4

pH of Exemplary Personal Care Formulations E4 and E5 Before and after Carbonation

The pH of each of Exemplary Formulations E4 and E5 (containing ASE rheology modifiers) as prepared in Examples 1 was measured before and after carbonation in Example 2. To visualize the change in pH, 3 droplets of hydroxynaphthol blue (available from Sigma-Aldrich) were added to the formulation prior to carbonation, at which time the formulation was a hazy pale pink color. After carbonation as described in Example 2, the formulation changed to opaque purple color indicative of a drop in pH. The pH was measured using a Schott Instruments CG 837 pH meter. The pH and observable rheology profile of each of Example Formulations E4 and E5 before and after carbonation is shown in Table 4.

TABLE 4
pH and Rheology Profile of Exemplary
Formulations Containing ASE Polymers

| | | |
|---|---|---|
| SAMPLE | E4 | E5 |

Prior to Carbonation

| | | |
|---|---|---|
| pH | 7.3 | 7.4 |
| Appearance | pink slightly hazy | pink hazy |
| | low viscosity gel | thick gel |

AFTER CARBONATION

| | | |
|---|---|---|
| pH | 5.9 | 5.1 |
| Appearance | purple opaque liquid | purple liquid |

As demonstrated by the results in Table 4, the pH and rheology profile of the formulations changed from a high viscosity gel-like formulation to a low viscosity liquid-like formulation after introduction of carbon dioxide and reduction of pH.

What is claimed is:

1. A personal care composition comprising:
(a) carbon dioxide;
(b) an ionic acrylic based rheology modifier comprising at least one of an alkali swellable emulsion polymer and a hydrophobically-modified alkali swellable emulsion polymer; and
(c) a neutralizer.

2. The personal care composition of claim 1, wherein the neutralizer comprises at least one of sodium hydroxide, potassium hydroxide, sodium carbonate, potassium carbonate, monoethanolamine, triethanolamine, aminomethylpropanol, aminomethylpropanediol, and tromethamine.

3. The personal care composition of claim 1, wherein the carbon dioxide is present in an amount of from 0.5 to 10 weight %, based on the total weight of the composition.

4. The personal care composition of claim 1, wherein the rheology modifier comprises a compound of Formula (I): 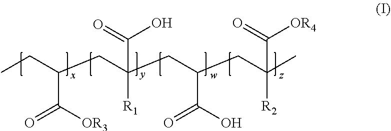
wherein
R₁ and R₂ are each independently H or a C₁-C₆ alkyl group;
R₃ and R₄ are each independently H or a C₁-C₂₄ alkyl or alkoxylated alkyl group; and
x, y, w, and z represent the percentage by weight that each repeating unit or derived monomer is contained within the polymer and add up to a total of 100 weight % of the total weight of the polymer, and x is from about 30 to 60 weight %, y is from 10 to 50 weight %, w is from 0 to 25 weight %, and z is from 0 to 25 weight %.

5. The personal care composition of claim 4, wherein each of R₁, R₂, R₃, and R₄ are H.

6. The personal care composition of claim 4, wherein R₁ and R₂ are each independently a C₁-C₆ alkyl group, and R₃ and R₄ are each independently a C₁-C₂₄ alkyl or alkoxylated alkyl group.

7. The personal care composition of claim 4, wherein the ratio of x:y is from 1:20 to 20:1, the ratio of x:w is from 1:20 to 20:1, and the ratio of x:z is from 1:1 to 500:1.

8. A method for altering over time the viscosity of a personal care composition comprising a rheology modifier and a neutralizer after release from a container in which the composition is stored, comprising:
(a) dissolving carbon dioxide in the composition in an enclosed container in an amount of from 0.5 to 10 weight %, based on the total weight of the composition; and
(b) topically administering an effective amount of the personal care composition from the enclosed container onto the skin or hair;
wherein the rheology modifier comprises at least one of an alkali swellable emulsion polymer and a hydrophobically modified alkali swellable emulsion polymer,
whereby after release from the container the viscosity of the composition changes as a function of a change in pH as dissolved carbon dioxide escapes.

9. The method of claim 8, wherein the rheology modifier comprises a compound of Formula (I): 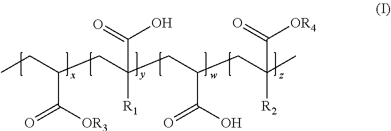
wherein
R₁ and R₂ are each independently H or a C₁-C₆ alkyl group;
R₃ and R₄ are each independently H or a C₁-C₂₄ alkyl or alkoxylated alkyl group; and
x, y, w, and z represent the percentage by weight that each repeating unit or derived monomer is contained within the polymer and add up to a total of 100 weight % of the total weight of the polymer, and x is from about 30 to 60 weight %, y is from 10 to 50 weight %, w is from 0 to 25 weight %, and z is from 0 to 25 weight %.
